(19) United States
(12) Patent Application Publication
(10) Pub. No.: US 2024/0288431 A1
(43) Pub. Date: Aug. 29, 2024

(54) FUNCTIONALIZED NANOPARTICLES

(71) Applicant: Lumito AB, Lund (SE)

(72) Inventor: Matthias Mickert, Lund (SE)

(21) Appl. No.: 18/553,908

(22) PCT Filed: Apr. 6, 2022
(86) PCT No.: PCT/EP2022/059159

§ 371 Date: Oct. 4, 2023

(30) Foreign Application Priority Data
Apr. 6, 2021 (SE) 2150428-7
Sep. 29, 2021 (SE) 2151192-8

(51) Int. Cl. G01N 33/574; G01N 33/532; G01N 33/533

(52) U.S. Cl. CPC G01N 33/57415; G01N 33/532; G01N 33/533; G01N 2333/912

(57) ABSTRACT

Nanocomposites are described, comprising an upconversion nanoparticle comprising at least one hydrophilic bifunctional linker that is coordinated to the UCNP and at least one functionalized antibody fragment, wherein the functionalized antibody fragment is covalently bound to the hydrophilic linker. Methods for preparing the nanocomposites and methods for the detection of target material in a biological sample using such nanocomposites are also described.

### FIELD

The disclosure relates to functionalized nanoparticles, in particular upconversion nanoparticles, and the use of such nanoparticles for detecting the presence of target analytes in biological samples, in particular in immunohistochemistry.

### INTRODUCTION

In histopathology, several techniques are used to study and analyse tissue, for instance, immunohistochemistry (IHC).

In immunohistochemistry, antigens, such as proteins and protein fragments, are detected in thin tissue sections. The antigens, which can also be carbohydrates or nucleic acids, are detected using labelled antibodies that bind to specific antigens in the biological tissue. IHC colouring or staining is a standard routine for diagnosing atypical cells, such as in tumour samples. Immunohistochemistry is also commonly used in basic research to understand the distribution and localization of biomarkers and differentially expressed genes and proteins in different parts of biological tissue.

The interaction between antibodies and antigens may be visualised in different ways. The most common practice is to conjugate the antibody to an enzyme, such as horseradish peroxidase, which can catalyze a chromogenic reaction leading to a detectable colour change in a sample. An alternative is to label the antibodies with a fluorophore, such as fluorescein, rhodamine, or Alexa dyes. The uses of fluorophores are restricted due to auto-fluorescence, i.e. fluorescence from the tissue itself, and require special preparations of the tissue sample to be useful. When using fluorophores, the conventional and preferred procedure based on formalin fixation and paraffin embedding suffers from even higher levels of auto-fluorescence than from the unprocessed tissue. In some cases, stains used in counterstaining in immunohistochemistry, such as haematoxylin and eosin, can absorb in a wavelength range that interferes with the excitation and/or emission of some reporters used for the detection of particular analytes. Eosin additionally shows yellow fluorescence upon irradiation with light in the blue to green, a disadvantage in fluorescence applications.

Photobleaching and cross-reactivity of different dyes, stains, and reports can lead to further limitations of this approach. These drawbacks have therefore limited the utility of immunofluorescence in the characterization and diagnostics of samples.

Upconversion nanoparticles, or UCNPs, are nanoscale particles with typical diameters of 10-100 nm that exhibit photon-upconversion, i.e. they absorb two or more photons of relatively low energy and emit one photon with higher energy. UCNPs typically absorb radiation in the infrared, with emission in the visible or UV regions, exhibiting anti-Stokes luminescence. The anti-Stokes process completely prevents autofluorescence from formalin-fixed paraffin-embedded tissue sections. UCNPs are typically composed of rare-earth-based lanthanide or actinide doped transition metals, such as NaYF₄:Yb/Er and NaYF₄:YbfTm.

UCNPs are chemically stable and can be functionalized by linking targeting ligands such as peptides, antibodies, and small-molecule drugs to the surface of the particles. These properties have been used in numerous applications, including in imaging (Liang et al J Nanobiotechnol 2020, 18:154).

Methods and reagents that show improved versatility in immunohistochemistry applications, including improved signal-to-noise ratio and increased sensitivity and specificity, thereby increasing the precision and quality of such analyses are highly desired. Additionally, the ability to simultaneously detect multiple targets would improve diagnostic accuracy and reduce analysis times.

### SUMMARY

The present invention provides nanocomposites and methods to overcome, eliminate or mitigate deficiencies of the prior art, for example, the deficiencies described above.

In an aspect, the invention relates to a nanocomposite material. The nanocomposite can in general comprise an upconversion nanoparticle (UCNP) that comprises a surface coating having at least one conjugation anchor point. The nanocomposite further comprises at least one functionalized antibody fragment, wherein the functionalized antibody fragment is covalently bound to the surface coating via at least one conjugation anchor point. The conjugation anchor point is therefore the structural location at which the functionalized antibody fragment is conjugated, or connected, to the UCNP.

The surface coating has physicochemical properties that render the upconversion nanoparticle water-dispersible, i.e. the surface coated nanoparticle can be dispersed in water or an aqueous solution. The surface coating serves multiple functions. First, the coating is attached to the UCNP, i.e. the coating is attached to the UCNP via non-covalent or covalent chemical interactions. Second, the coating has anchor points, to which a functionalized group such as an antibody fragment can be conjugated (i.e. bonded or connected). Third, the coating should have hydrophilic properties, so that the modified nanoparticle becomes dispersible in water.

The surface coating can comprise at least one linker that is coordinated to the UCNP. The linker is preferably at least bifunctional, i.e. the linker contains at least one functional moiety or group that can interact with the UCNP and at least one additional chemical entity that can be used for further reactions.

To aid in the water-dispersive properties of the nanocomposite, the linker can be hydrophilic or at least contain a hydrophilic portion (i.e., the linker is at least partly hydrophilic) that renders the final construct dispersible in water or aqueous solutions.

The term “composite”, in the present context, shall be understood as representing a material that is produced from two or more constituent materials that each has dissimilar chemical and/or physical properties to create a material that has properties that are at least partially unlike the individual elements. It follows that the term “nanocomposite” in the present context shall be understood to represent a composite material that is of a small unit size that is in the range of about 1 to about 100 nm.

The term “hydrophilic”, in the present context, shall be understood to describe a molecule or molecular entity that tends to dissolve in water, because its interactions with water are more thermodynamically favorable than its interaction with oil or other hydrophobic solvents.

Antibody fragments include, without limitation, Fab fragments, F(ab)₂ fragments, Fab′ fragments, F(ab′)₂ fragments, Fd fragments, Fd′ fragments, Fv fragments, 61-residue subdomains of the antibody heavy-chain variable domain, also known as minibodies, and domain antibodies (dAbs). Antibody fragments can generally be made by conventional procedures, such as proteolytic fragmentation procedures, for example as described in J. Goding, Monoclonal antibodies; Principles and practice 98-118 (1984).

Preferably, the antibody fragment contains only one disulfide bond that can be specifically reduced to generate free sulfhydryl groups. In the case where the antibody fragment contains a single disulfide bond, two adjacent thiol groups can thus be released by reducing the disulfide bond in the antibody fragment. Thus, it should be possible to reduce the disulfide bond(s) in a specific manner to release free sulfhydryl groups that can be used for downstream chemical modification or attachment.

The surface coating can generally comprise one or more coordinating moieties that tether the coating to the nanoparticle. The coordinating moiety can for example be a carboxyl or dicarboxyl moiety, a phosphate or bisphosphate moiety, or a phosphonate or bisphosphonate moiety. The tether can bind to the nanoparticle via non-covalent or covalent interaction, preferably via non-covalent coordination to the particle surface.

Phosphonate functionalization of upconversion nanoparticles has been described in the art (Alonso-de Castro et al, Inorganics 2019 60 doi:10.3390; Li et al, ACS Nano 2015, 9:3293). Suitable phosphonates include salts of etidronic acid, alendronic acid, neridronic acid, nitrilotri(methylphosphonic)acid, N-(phosphonomethyl)iminod iacetic acid (PMIDA), 3-bromopropyl)phosphonic acid (BPPA), (aminomethyl)phosphonic acid (AMPA), ethylenediamine tetra(methylene phosphonic acid) (EDTMP). The negatively charged phosphonates strongly coordinate to lanthanide ions at the particle surface, making them suitable to functionalize the UCNPs.

The phosphonate moiety is conveniently connected to a hydrophilic linking moiety such as a polyetheleneglycol (PEG) molecule which acts as a linker between the nanoparticle and fragmented antibody. The linker can comprise a phosphonate or bisphosphonate decorated polyethylene glycol (PEG)ₙ having a click reactive group on the opposite of the phosphonate group, n represents the number of ethylene glycol groups in the PEG and where n is an integer greater than or equal to 3. For example, n can range from 3 to 150, from 5 to 150, from 10 to 150, from 20 to 150, from 30 to 150, from 40 to 150, or from 50 to 150, as in for example alkyne-(PEG)ₙ-neridronate.

Alternatively, the surface coating can comprise a click reactive (PEG)ₙ moiety (such as (PEG)ₙ-azide), wherein the UCNP is modified on its surface with amine-functionalized silica or carboxide-functionalized silica or a polymer having free carboxyl or amino groups such as poly(acrylic acid). The (PEG)ₙ moiety is bound to the functionalized silica/polymer, leaving the free click reactive group for reaction with a functionalized antibody fragment.

The linker can alternatively comprise a protein or peptide molecule to which a click reactive group is attached. A “click reactive group”, as defined herein, refers to a chemical moiety that can react with a second click reactive group to form a covalent bond between the two groups, thereby leading to conjugation of the two molecules to which the click reactive groups are connected. Further options include hydrophilic polymers such as poly(acrylic)acid that contain are modified with a click reactive group.

In general, any suitable click chemistry known in the art can be used to react the functionalized antibody fragment and the functionalized UCNP. For example, the click chemistry can include azide-alkyne cycloaddition chemistry between a free azide group and alkyne-containing groups such as bicyclo[6.1.0]non-4-yne (BCN), carboxymethylmonobenzocyclooctyne (COMBO), dibenzo-fused cyclooctynes (DBCO/DIBAC), biarylazacyclooctynone (BARAC), 4,8-diazacyclononyne (DACN), difluorocyclooctyne (DIFO, DIFO2, DIFO3), dimethoxyazacyclooctyne (DIMAC), aryl-less octyne (ALO), nonfluorinated cyclooctyne (NOFO), TMTH-Sulfoxlmine (TMTHSI; CliCr), monofluorinated cyclooctyne (MOFO) and cyclooctyne (OCT).

The click chemistry can alternatively involve an inverse electron-demand Diels-Alder reaction between trans-cyclooctene and tetrazine.

### Alkynes for Strain-Promoted Azide-Alkyne Clycloadditions

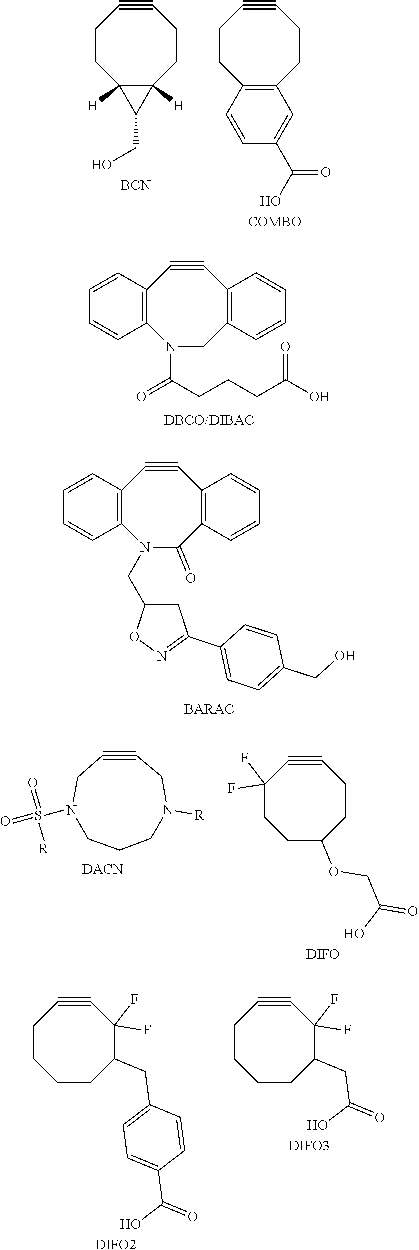

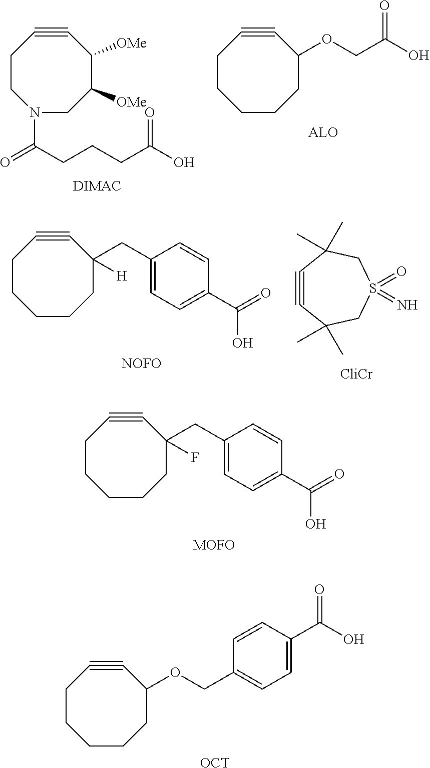

### Inverse Electron-Demand Diels-Alder Reaction

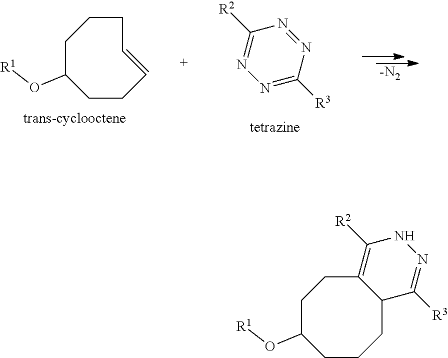

In general, the two click reagents can be conveniently provided on either the functionalized UCNP or the functionalized antibody fragment. For example, in chemistry involving azide-alkyne cycloaddition, the azide group can be provided on either the functionalized UCNP or the functionalized antibody fragment. It follows that the second click reagent is provided on the functionalized UCNP or antibody fragment that has not been functionalized with an azide group.

The nanocomposite can have the general formula:

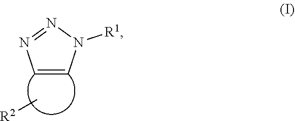

where R² represents antibody fragment-(PEG)ₙ, wherein n=1-12, preferably 4-12, and R¹ represents UCNP-Y-(PEG)n, wherein Y is a phosphonate or bisphosphonate group and n=3-150, such as 10-150, or 50-150, wherein the ring structure to which R² is attached is an optionally substituted cyclic or heterocyclic ring structure containing from 5 to 9 atoms; or where R² represents UCNP-Y-(PEG)ₙ, wherein n=1-12, preferably 4-12 and Y is a phosphonate or bisphosphonate group, and R¹ represents antibody fragment-(PEG)ₙ, wherein n=3-150, such as 10-150, or 50-150, wherein the ring structure to which R² is attached is a substituted cyclic or heterocyclic ring structure containing from 5 to 9 atoms.

The ring structure to which R² is attached can contain two or more further substituents that connect to the ring structure to form a tri- or tetracyclic fused ring structure, wherein at least one of the rings, and optionally at least two of the rings, in the thus fused ring structure contain at least one heteroatom.

The nanocomposite can alternatively have the general formula:

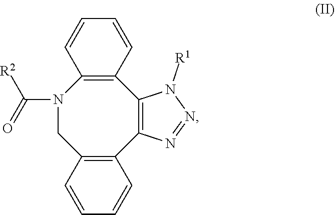

where R² represents antibody fragment-(PEG)ₙ, wherein n=1-12, preferably 4-12, and R¹ represents UCNP-Y-(PEG)n, wherein Y is a phosphonate or bisphosphonate group and n=3-150, such as 10-150, or 50-150; or where R² represents UCNP-Y-(PEG)ₙ, wherein n=1-12, preferably 4-12 and Y is a phosphonate or bisphosphonate group, and R¹ represents antibody fragment-(PEG)n, wherein n=3-150, such as 10-150, or 50-150.

The nanocomposite can be prepared by reacting a functionalized antibody fragment (e.g., Fab) and a suitably coated UCNP that contains a reacting spacer group that can bind to the functionalized antibody. In a second aspect, the invention, therefore, provides a method for the preparation of a nanocomposite, comprising a step (a) of providing an upconversion nanoparticle comprising a multifunctional linker on its surface, the multifunctional linker having a free click reactive (e.g., azide) moiety. In the following step (b) an antibody fragment having at least one pair of adjacent thiol groups is reacted with thiol reactive reactive moiety (e.g., a bis-sulfone containing moiety), whereby the adjacent thiol groups react with the thiol reactive moiety to form a functionalized antibody fragment. The upconversion nanoparticle comprising the multifunctional linker is subsequently reacted with the functionalized antibody fragment in a click reaction to form the nanocomposite having the functionalized antibody fragment covalently bound to the multifunctional linker.

The multifunctional linker can for example be bifunctional. The multifunctional linker can preferable be hydrophilic or contain hydrophilic portions (i.e., the linker is at least partly hydrophilic) so that the thus modified UCNP becomes dispersible in water or an aqueous solution.

The adjacent interchain thiol groups can represent a reduced form of the antibody fragment, obtained or obtainable by reduction of the corresponding interchain disulfide bond between the two thiol groups. The antibody fragment can preferably contain a single interchain disulfide bond, thereby releasing a single pair of adjacent thiol groups upon reduction of the disulfide bond. Accordingly, the term “adjacent thiol groups”, as disclosed herein, refers to thiol groups that are released upon reduction of a disulfide bond in the antibody fragment.

The nanocomposites comprise luminescent UCNP particles that may, in general, emit Stokes or anti-Stokes shifted light when excited at particular wavelengths. For example, particles may emit anti-Stokes shifted light (such as visible light) when excited at a particular wavelength, such as infrared or near-infrared light, for example at 980 nm. Additionally, and/or some upconverting particles may also emit Stokes shifted fluorescent light at a longer wavelength in the infrared or near-infrared light, such as around 1500 nm. This light may also be used as it is outside of the wavelength range of auto-fluorescent light emitted from the background, such as from the biological sample itself, like tissue, or from the fixation and embedding of the sample. In some examples, emitted Stokes and anti-Stokes shifted light from the same particles may be used when analysing the sample.

The UCNP particle can be selected from such particles that are known in the art. One of the brightest types of upconversion nanoparticles consists of a NaYF₄ host lattice that is doped with the sensitizer Yb³⁺ and emitting ions like Er³⁺ or Tm³⁺. UCNPs are excited at around 980 nm followed by emission of light with shorter wavelength (see e.g., Chen et al. Chem Rev 2014, 114:5161 for a review). The near-infrared excitation minimizes light scattering and completely prevents autofluorescence of biological matrices, a distinct advantage in imaging applications of biological tissues using UCNPs.

The anti-Stokes shift in the luminescent radiation from the UCNPs provides an improved signal-to-background ratio compared to conventional fluorophores, as the auto-fluorescence from the tissue can be eliminated. Paraffin-embedded tissue has a strong background auto-fluorescence that makes it hard to use conventional fluorophores as labels without time-consuming and costly preparations of the samples. Hence, apart from improving the contrast, luminescent particles, such as upconverting particles, save time and cost as conventional formalin fixation and paraffin embedding can be used. The labelled target moiety or probes may be used in histology, such as histopathology and particular immunohistochemistry, or cytology, such as cytopathology and in particular immunocytochemistry, or hybridizations, such as in situ hybridization (ISH), in combination with counterstaining, such as standard counterstaining used in histology, and in particularly immunohistochemistry, cytology, and in particularly immunocytochemistry, or hybridizations, such as in situ hybridization (ISH).

In another aspect, the invention relates to a method for the detection of target material in a biological sample, such as a tissue sample or a blood sample. The method can comprise steps of providing a biological sample and contacting a nanocomposite comprising at least one upconversion nanoparticle linked to at least one functionalized antibody fragment with the biological sample. Upon contact, the nanocomposite selectively binds to the target material in the biological sample. Imaging can then be performed on the sample to obtain an imaging signal from the nanocomposite to thereby identify the target material.

The target material can be any analyte or biological molecule that is recognized by an antibody fragment. The target material can be endogenous, i.e. a target material from the biological sample itself, such as antigens, antibodies, proteins, cell membrane components, carbohydrates, or nucleic acids (e.g., DNA or RNA). The target material can also be an antibody (e.g., a primary antibody) that is recognized by the antibody fragment and in turn, binds specifically to an epitope in the biological sample.

The biological sample can be a tissue sample that has been fixed, e.g. by formalin fixation and paraffin embedding. The tissue sample can also be a frozen tissue sample.

The method can be adapted to detect multiple targets. In such multiplexing applications, multiple nanocomposites comprising different antibody fragments with different affinities can be used to detect different target moieties. Each nanocomposite can have a unique luminescence emission spectrum. Thereby, each nanocomposite, and therefore each target being recognized by the nanocomposite, can be detected by capturing the signal at a wavelength characteristic for each nanocomposite. In such applications, excitation can be performed at a single wavelength (e.g., 980 nm), and the emission is captured at the wavelength characteristic for the UCNP in question.

Alternatively, nanocomposites can be generated wherein each UCNP contains more than one type of antibody fragment. Thereby, the multiplexing is a functionality of each UCNP rather than a functionality of using variety of different UCNPs.

Such multiplexed constructs can be generated for example by using a mixture of functionalized antibody fragments in the conjugation step to the UCNP. The antibody fragments can be modified using the same functionalities, i.e. the same chemistry. The antibody fragments can also be functionalized differently, i.e. using different functionalities (different chemical modifications) for each antibody fragment.

The stoichiometry of reagents and/or the chemistry (nature of functionalization of antibody fragments) can be used to generate nanocomposites containing multiple antibody fragments with a specifically designed composition. In other words, the stoichiometry of the different (two or more) antibody fragments decorating each UCNP can be varied as desired.

The UCNP can alternatively be functionalized with two antibody fragments with different functionalities, such as an anti-mouse antibody fragment and an anti-rabbit antibody fragment. This way, a UCNP is generated that can be used for use with either mouse or rabbit antibodies.

Images can be captured using upconversion microscopy. There can be a step of counterstaining the biological sample (e.g., tissue sample) that either precedes or follows detection of the nanocomposite. Standard counterstains may be chromogenic or based on fluorescence. The chromogenic and/or fluorescent dyes or stains may either be non-specific, staining most of the cells in the same way or specific, selectively staining particular organelles or cellular compartments or chemical molecules within cells/tissues, such as the nucleus by targeting nucleic acids, cell walls or membranes. Thereby, imaging can be performed on the same tissue material that detects most of the cells and/or particular organelles or compartments and/or specific molecules, and imaging that is specific for certain target moieties using the nanocomposites.

### BRIEF DESCRIPTION OF THE DRAWINGS

FIG. 1 shows an example of a conjugation procedure of an upconversion nanoparticle to an antibody fragment as described herein.

FIG. 2 shows a schematic overview of a labelling workflow using the nanocomposite described herein.

FIG. 3 shows images of a labelling using an upconversion nanoparticle—antibody fragment fusion construct bound to an anti-HER2 primary antibody. In (A) is shown labelling of HER2 on a cancer cell pellet, in (B) labelling of HER2 in breast cancer tissue.

FIG. 4 shows images from a duplex labelling of two different primary targets (HER2 and PR) using two different upconversion nanoparticle—antibody fragment fusion constructs bound to primary antibodies. In (A) is shown signal from green-emitting nanocomposite bound to an anti-HER2 antibody; in (B) signal of nanocomposite emitting at 800 nm bound to an anti-PR antibody; in (C) is shown an overlay of the images in (A, green) and (B, blue).

FIG. 5 shows results of gel electrophoresis of Fab and Fab-DBCO constructs. (lane 1) Fab; (lane 2) reduced Fab; (lane 3) Fab after mild reduction; (lane 4) Fab after additional reduction using DTT; (lane 5) Fab-DBCO purified in Tris buffer; (lane 6) Fab-DBCO after reduction with DTT; (lane 7) Fab-DBCO after purification in HEPES; (lane 8) Fab-DBCO after reduction with DTT.

FIG. 6 shows an absorption spectrum of an unpurified and purified reaction mixture after DBCO modification of the Fab.

FIG. 7 shows an FTI-IR analysis of the coating of UCNPs by azide-PEG-neridronate/alendronate.

FIG. 8 shows a magnetic bead assay used to confirm the successful conjugation of UCNPs with Fab. A) Magnetic beads coated with arabbit IgG form an immunocomplex with anti-rabbit UCNP-Fabs. B) Control experiment using magnetic beads coated with an mouse IgG antibody. C) Magnetically purified samples (1-8) imaged with an upconversion microscope. Low intensities in the control (lower row) confirm that the anti-rabbit UCNP-Fabs bind specifically to the rabbit antibody-coated magnetic beads.

FIG. 9 shows the Dynamic Light Scattering (DLS) of anti-mouse Fab-conjugated UCNPs.

FIG. 10 shows the DLS of anti-rabbit Fab UCNPs.

### DESCRIPTION

The present invention relates to constructs containing antibody fragments connected to upconversion nanoparticles (UCNP) and the use of such constructs in histology, in particular histopathology or immunohistochemistry. The skilled person will appreciate that the constructs and methods disclosed herein will also be advantageous in cytology, such as cytopathology, and immunocytochemistry. Further advantages include the use in hybridization, in particular in situhybridization, such as fluorescence in situ hybridization (FISH). However, the skilled person will appreciate that the following description is not limited to these applications, the constructs and methods described herein can be applied to other samples and analyses and used with alternative moieties and targets.

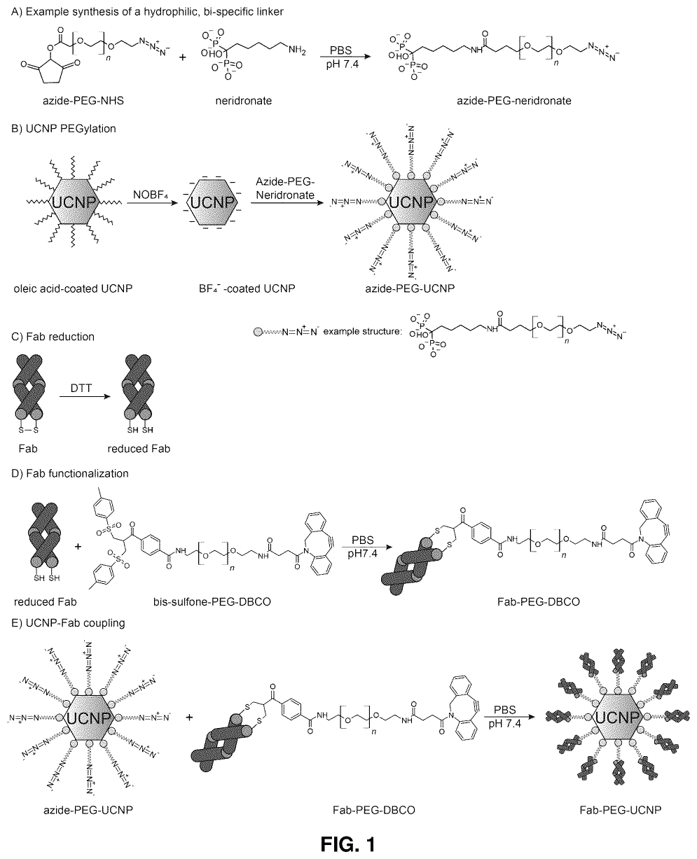

In FIG. 1 an overview of a procedure for preparing and conjugating antibody fragments (Fab) to the UCNP is shown. In step (A) there is shown an example of the preparation of a linker moiety, in which an N-hydroxylsuccinimide (NHS) functionalized polyethylene glycol (PEG) containing an azide moiety at its free end (azide-PEG-NHS) is reacted with neridronate via the terminal amino group of neridronate to form a bispecific azide-PEG-neridronate fusion having a free azide group at its end.

Azide-PEG-NHS constructs are known in the art. The azide-PEG-NHS constructs can in general include any suitable PEG length, i.e. (PEG)ₙ, where n ranges from about 3 to 150 or greater. Any particular construct typically has a single length (PEG)ₙ, i.e. n takes on a single value for the particular construct, e.g. (PEG)₇₀. It can be preferable to use (PEG)ₙ with values for n in the range of about 40 to about 150, about 50 to about 150, about 50 to about 130, about 60 to about 130 or about 60 to about 120. The linker group should provide the UCNP with good water dispersion properties and protect the particles from dissolution, while at the same time provide a uniform coating that also reduces non-specific binding of the UCNP.

The construct has a coordinating moiety, here exemplified by neridronate, that binds noncovalently to the UCNP. In general, the coordinating group can be any group capable of coordinating to UCNPs. Examples include carboxyl or dicarboxyl moieties, phosphate or bisphosphate moieties, or phosphonate or bisphosphonate moieties.

The linker moiety can then be coordinated to the UCNP to provide the UCNP with a surface coating of the linker moiety. In (B), this step is illustrated starting from an oleic-acid coated UCNP, which is first stripped off via a ligand exchange reaction with nitrosonium tetrafluoroborate (NOBF₄). The weakly coordinating NOBF₄ is replaced by the more strongly coordinating azide-PEG-neridronate to form a UCNP having a coating of azide-PEG-UCNP on its surface.

The azide-PEG-neridronate coated UCNPs have a near-neutral surface charge that is advantageous, since it minimizes electrostatic interactions, for example, non-specific attachment onto glass slides that are commonly used in immunohistochemistry applications. PEG is also known to generally reduce the non-specific binding of UCNPs (Anal. Chem. 2019, 91, 15, 9435-9441).

The antibody fragment is functionalized via its interchain thiol groups. As shown in (C), the thiol groups are released using a reducing agent such as dithiothreitol (DTT) or Tris(2-carboxyethyl)phosphine (TCEP) that reacts with exposed disulfide bridges. Thereby, two thiol groups that are adjacent in space are released and available for subsequent functionalization.

Functionalization of the thiol groups is done with a bifunctional thiol-reactive reagent that on one end has a functional moiety that can connect to the adjacent thiol groups and the other end has a second functional moiety that can react with the coated UCNP to generate the final UCNP-antibody fragment construct.

An exemplary reagent is a bis-sulfone-containing reagent, such as bis-sulfone-(PEG)ₙ-DBCO, as shown in (D). Such reagents are known in the art, enabling site-specific incorporation of a DBCO (dibenzocyclooctyne) moiety onto a target protein, such as antibodies.

A distinct advantage of functionalizing the antibody fragment with a thiol-reactive reagent such as bis-sulfone-PEG-DBCO is that a 1:1 ratio of the antibody fragment to DBCO is ensured. This is critical since it reduces the aggregation of UCNP in the subsequent Click reaction. Further, the nature of the functionalization group, where binding to the antibody fragment and binding to the UCNP are on opposite ends of the functionalization group, ensures that the conjugation to the UCNP does not interfere with the antibody fragment activity. The intervening PEG spacer on the functionalization group also serves the role of minimizing steric hindrance during the coupling reaction to the UCNP, increasing the reaction yield.

In particular, for antibody fragments containing a single disulfide bond, the functionalizing via the two adjacent thiol groups that are released upon reduction leads to a controlled functionalization reaction, which also minimizes aggregation issues upon subsequent click reaction chemistry. The disulfide bond can preferably be an interchain disulfide bond.

The coupling reaction of the functionalized antibody fragment to the azide-PEG-UCNP is shown in (E). No catalyst (such as Cu(I)) is required for the reaction, which is advantageous due to the cytotoxic activity of copper, and also due to the fact that copper can lead to a loss of activity of the antibody fragment. Further, reactive intermediates that are formed during the Cu-catalyzed Click reaction with L-ascorbate as reducing agent are avoided, thereby protecting the integrity and activity of the antibody fragment. Downstream applications of the UCNP-antibody fragment construct thereby will have improved sensitivity.

As described herein, any suitable click reaction reagents can be introduced via functionalization of the UCNP and the antibody fragments, the coupling of an antibody fragment to the UCNP being carried out via the two reactive components of the click reaction. Suitable click reagents are known in the art, some of which are described further herein.

Click reactions offer several advantages especially over frequently used EDC/NHS chemistry. 1) Click reactions are highly specific between azide and alkyne groups, both of which are typically not present in natural biomolecules or as impurities. In EDC/NHS chemistry the reaction occurs between activated carboxyl groups on the UCNP and primary amino groups on the coupling partner. Antibody fragments are proteins that contain several amino functionalities. This can cause nanoparticle aggregation because two or more nanoparticles can react with the same antibody fragment and form bigger networks (aggregates) that lower the performance of IHC applications. Additionally, amine impurities can reduce the reaction yield in EDC/NHS-based reactions. 2) The click reaction chemistry presented here ensures controlled coupling of the modified antibody fragment to the UCNP, with the antibody fragment-linker moiety sticking out from the UCNP particle to generate a nanocomposite with the antigen binding site of the antibody fragment pointing away from the UCNP in an exposed manner, ensuring their functionality. In EDC/NHS chemistry, fragmented antibodies can bind to the UCNPs via amino groups present at the antigen binding site causing the antibody to lose its binding ability. 3) Click reactions are very mild reactions that take place under physiological conditions (pH 7.4). In EDC/NHS chemistry the carboxylated nanoparticles are activated in a first step with a mixture of EDC and NHS typically in MES buffer at a slightly acidic pH of around 6.2. The reaction mixture needs to be purified quickly to remove excess of EDC/NHS and is then transferred to a buffer with physiological pH containing the antibody fragment.

UCNP-antibody constructs have been used for immunohistochemistry applications. There are however several nonobvious advantages of using antibody fragments that have antigen-binding activities compared with the use of either full antibodies or the use of typical streptavidin conjugates.

For example, antibody fragments have a molecular weight that is much lower (about one third) than full antibodies. This leads to a larger number of antibody fragments that can bind to the UCNP, increasing the sensitivity of using such constructs. Furthermore, increased tissue penetration capabilities are expected when using antibody fragments compared with full antibodies, improving sensitivity by reducing steric hindrances. Another advantage is the lack of the Fc domain, which reduces non-specific binding, again increasing the sensitivity and specificity of IHC applications.

Yet another advantage is that multiplexing applications are conveniently achieved using antibody fragments. Thus, as illustrated in Example 2 herein, by using multiple antibody fragment-UCNP constructs that recognize different epitopes, it becomes possible to detect different targets, for example different cell types. Such applications can be done by using primary antibodies that specifically detect different epitopes, and detecting the primary antibodies using selective secondary antibody fragment-UCNP constructs. Irradiation of UCNPs in multiplex applications can be done at a single wavelength, with emission of luminescent radiation being specific for each UCNP. In both cases, detection is done at wavelengths with little or no interference from background autofluorescence, which is a general and well-known advantage of using upconversion nanoparticles in IHC applications.

IHC analysis can be performed on samples that contain fixed cells. The sample can be any sample suitable for pathological examination, which has been fixed using an appropriate fixative. Exemplary fixatives include, without limitation, formalin and Bouin's solution, and the use thereof is well known in the art. The fixed sample is typically embedded in an embedding media such as paraffin or resin. The sample to be used in carrying out the methods of the present invention may be cut into sections and mounted on slides that are suitable for microscopic examination, e.g., glass or plastic slides. No deviation from the standard protocol for pathology specimen handling is required to prepare the samples for analysis. Suitable specimens include, without limitation, tissue samples or biopsies, organ resections, and fluid samples.

The tissue sample can be any sample that contains cells of interest, in which case the sample can be a fluid sample or a solid tissue sample. Although the invention is not limited to cancer cell detection, the analysis of cancerous tissue, or tissue suspected of being cancerous or containing cancerous cells, represents one suitable utility thereof. Thus, tissue specimens can include specimens of solid tumors found in all non-hematopoietic sites, including, but not limited to lung, breast, colon, and entire gastrointestinal tract, prostate, brain, pancreas, and skin. The tissue specimen can also include non-solid tumors, e.g., lymphomas, leukemias, and plasma cell neoplasms, and involves the analysis of patient tissues that include all hematopoietic organs, without limitation, blood, lymph nodes, tonsil, spleen, thymus, and bone marrow.

IHC analysis using the nanocomposite particles disclosed herein can include detecting specific cell populations in tissue samples containing heterogeneous populations of cells. Such analysis can include providing a sample containing population of cells, for example in the form of a tissue sample comprising fixed cells. The tissue sample, which can suitably be a formalin-fixed paraffin-embedded tissue, is subsequently sectioned and prepared for imaging using the antibody fragment UCNPs described herein.

In the analysis, a primary antibody recognizing the first antigen in or on cells in a sample to be analyzed is used in combination with the nanocomposite. Thus, a first epitope is recognized by the antibody, provided by a protein or peptide of interest that is in or on a cell in a tissue sample to be analyzed. An antibody fragment covalently bound to a hydrophilic linker attached to an upconverting nanoparticle is subsequently used to detect the antibody via a second epitope on the primary antibody. Specific detection of the target cells is therefore provided by the first antibody, which is recognized by the nanocomposite containing an antibody fragment.

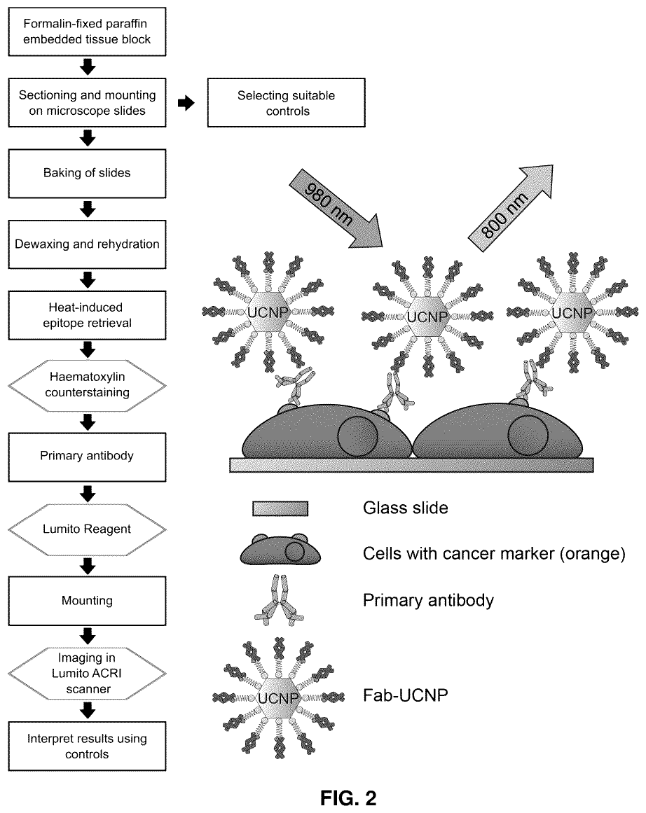

In FIG. 2 there is shown an exemplary workflow for the labelling and detection of a tissue sample to be analyzed. In this example, the analysis starts with a formalin-fixed paraffin-embedded tissue sample. The sample is sectioned and mounted on microscope slides. Negative control samples can be selected as appropriate from the tissue block being analyzed. The sections on the slides are treated using standard methods, e.g. by baking, dewaxing/rehydration, and heat-induced epitope retrieval.

In the example shown, there is a step of haematoxylin counterstaining performed before the treatment with the nanocomposite. The skilled person will appreciate that the counterstaining step is optional but can provide useful information when comparing or overlaying images obtained from the counterstaining and the UCNPs.

The counterstain can in general be a chromogenic or fluorescent stain or dye. Images of the counterstain can in general be obtained before or after the nanocomposite labelling of the sample.

The sample is subsequently treated with a primary antibody that binds specifically to an epitope target in the sample, for example, an exposed cell surface marker. The use of a primary antibody is optional, as the method can in general be performed by direct treatment with the antibody-fragment containing nanocomposite.

In the protocol shown in FIG. 2, the primary antibody is detected using the nanocomposite, indicated by Fab-UCNP. The nanocomposite binds specifically to the antibody, which in turn is specifically bound to its target on the cell surface. Image analysis of the resulting complex is performed by irradiating the sample with light in the near-IR region (for example 980 nm), with luminescence being detected at 800 nm. The methods can be easily adapted for multiplexing, whereby multiple different target moieties (e.g., different cell types, different receptors/proteins, etc) can be detected in the same tissue sample. This can be done by generating nanocomposite constructs that have different antibody fragments connected to UCNPs with different luminescence characteristics, i.e. emission at different wavelengths. The nanocomposites, for example, two different nanocomposites, can subsequently be used to detect different targets in a tissue sample, such as a formalin-fixed paraffin-embedded tissue sample. Results of imaging using each nanocomposite can subsequently be overlayed if desired to for example compare signal intensity, signal distribution, and/or signal location in the tissue. Such applications can have a variety of applications, for example in the diagnosis of tissue samples suspected of containing cancerous growth or other cellular malignancies or cellular changes.

Alternatively, different primary antibodies can be used to detect different primary targets in the tissue sample, and the different primary antibodies are detected using different nanocomposites, each being specific for one antibody used in the assay.

As will be appreciated from the foregoing, the nanocomposites described herein, their preparation, and their use in imaging methods provide several advantages over the prior art. These advantages include for example the following:

| Process | Advantages |
|---|---|
| PEG linker | Reduces non-specific binding |
| | Yields highly uniform particles compared to other methods like |
| | silanization or polymer coating with (poly)acrylic acid |
| | Particles have a near-neutral surface charge preventing them from |
| | electrostatic attachment onto cationized glass slides commonly |
| | used in IHC |
| | Particles are rendered highly water dispersable |
| | PEGylation protects the nanoparticle from dissolution |
| Fab | Ensures a 1:1 ratio of Fab to DBCO, drastically reducing the |
| functionalization | aggregation of the UCNPs during Click reaction |
| with bis-sulfone- | The bis-sulfone-PEG-DBCO specifically binds on the opposite |
| PEG-DBCO | location of the antigen-binding-site, preserving the Fab activity |
| | The PEG-spacer reduces steric hindrances during the UCNP- |
| | coupling increasing the reaction yield |
| Copper-free | No argon/nitrogen purging necessary to prevent foam formation of |
| Click reaction | the sample prevents denaturation of Fabs on liquid/air interface |
| | Simplified manufacturing process compared to Cu-catalyzed Click |
| | No cytotoxic Cu²⁺ needed |
| | Omitting Cu addition prevents activity-loss of Fabs caused by the |
| | interaction of Cu²⁺ with the Fab |
| | Reactive intermediates formed during Cu-catalyzed Click reaction |
| | with L-ascorbate as a reducing agent are omitted, protecting Fab |
| | activity |
| Use of Fabs | Fabs have ⅓ the molecular weight of a full antibody which |
| compared to full | increases the number of Fab molecules that can bind onto a UCNP |
| antibodies or | The smaller size of Fabs improves the tissue penetration in IHC |
| streptavidin | experiments |
| | Lack of Fc fragment reduces non-specific binding |
| | Opens the possibility for multiplexed applications compared to |
| | streptavidin conjugates |
| | Better control of whole conjugation process compared to full |
| | antibodies increasing reproducibility |

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to” and are not intended to exclude other components.

Those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure are used. The present invention also covers the exact terms, features, values, and ranges, etc. in case these terms, features, values, and ranges, etc. are used in conjunction with terms such as about, around, generally, substantially, essentially, at least, etc. (i.e., “about 3” shall also cover exactly 3 or “substantially constant” shall also cover exactly constant). The term “at least one” should be understood as meaning “one or more”, and therefore includes both embodiments that include one or multiple components. Furthermore, dependent claims refer to independent claims that describe features with “at least one” have the same meaning, both when the feature is referred to as “the” and “the at least one”.

Features disclosed in the specification unless stated otherwise, can be replaced by alternative features serving the same, equivalent, or similar purpose. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination. The scope of the disclosure is only limited by the appended patent claims.

The invention is further described by the following non-limiting examples.

### Example 1

### Detection of Single Nanocomposite in Cancer Tissue

Human Epidermal Growth Factor Receptor 2 (HER2) has been shown to be important in the development and progression of aggressive cancer types. The protein can be targeted by antibodies, for example, the monoclonal antibody trastuzumab, also called Herceptin.

HER2 was detected by visualizing the luminescence of antibody fragment (Fab) containing UCNPs (Fab-UCNPs) specifically bound to an anti-HER2 primary antibody in a cell pellet from a cancer cell line (BT474 FFPE) and in breast cancer tissue. Results are shown in FIG. 3. In (A) there is shown labelling of HER2 on the BT474 FFPE cell pellet, and in (B) labelling of HER2 in breast cancer tissue is shown. As can be seen, clear labelling is obtained with high contrast.

### Example 2

### Detection of Two Nanocomposite in Cancer Cells

Simultaneous detection of two cancer markers was performed by visualizing the luminescence of Fab-UCNPs specifically bound to primary antibodies. Two different nanocomposites were used to label BT474 FFPE cell pellets. One of the nanocomposites has a green luminescence signal, while the other has luminescence at 800 nm. Both nanocomposites are excited at the same wavelength. Images were subsequently taken at the same position on the cell pellet.

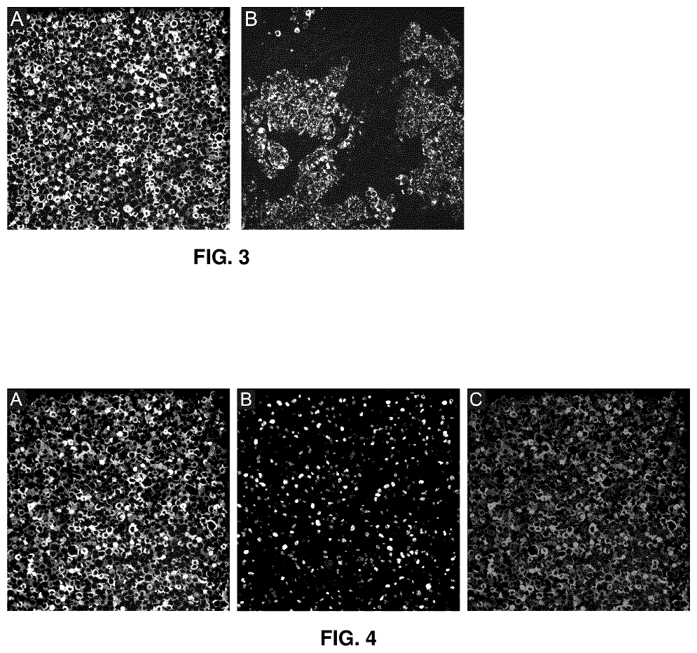

Results are shown in FIG. 4. In (A) there is shown a luminescent signal of green-emitting Fab-UCNPs bound to a primary anti-HER2 antibody, while in (B) luminescent signal of Fab-UCNPs emitting at 800 nm that bound to an anti-PR antibody on a BT474 FFPE cell pellet is shown. In (C) there is shown an overlay of A (green, HER2) and B (blue, PR) in pseudocolors. As can be seen, labelling is clear and distinct, with the two signals readily distinguishable in the overlay.

### Example 3

### Analysis of Fab and Fab-DBCO Conjugates

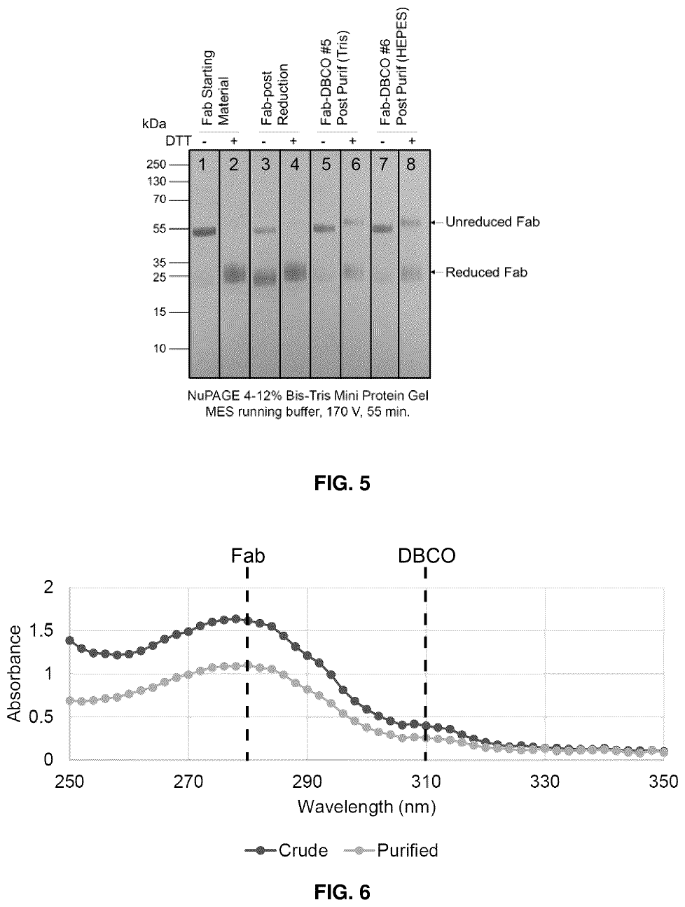

Reduction of Fab and Fab-DBCO conjugates was assessed by gel electrophoresis, as shown in FIG. 5.

Unmodified Fab molecules (lane 1) migrate in the gel and give a strong band at a molecular weight of ˜50 kDa. If the Fab antibody is reduced by a reducing agent like DTT (lane 2) the Fab is split into two smaller units that have half of the mass of the Fab antibody resulting in a band at ˜25 kDa. Lane 3 shows the Fab antibody after mild reduction as performed before coupling to the DBCO reagent and lane 4 the reduced Fab with additional reduction by DTT. The band from lane 5-8 show Fab-DBCO conjugates. In lane 5 the band of Fab-DBCO is visible at ˜50 kDa after addition of DTT (lane 6) not all of the Fab-DBCO molecules are reduced because the rebridged Fab antibodies are less prone to reduction compared to free Fab antibodies. Lane 7-8 display a functionalized Fab that was purified in HEPES buffer.

### Example 4

### Determination of Fab/DBCO Ratio

The successful functionalization of Fab fragments with click active DBCO was confirmed via absorption spectroscopy. Results are shown in FIG. 6. The upper curve shows the absorption spectrum of the unpurified reaction mixture and the lower curve the absorption spectrum after purification. The Fab fragment absorbs light at 280 nm while DBCO absorbs at 310 nm. By calibrating with known concentrations of Fab and DBCO the ratio of the two components can be calculated. Typical values for a Fab:DBCO ratio is 0.93-0.97 proving that Fab molecules are functionalized with not more than 1 DBCO molecule.

### Example 5

### Confirmation of UCNP Coating

Fourier-transformed infrared spectroscopy (FT-IR) was used to confirm the coating of UCNPs by azide-PEG-neridronate/alendronate. A calibration curve using pure azide-PEG was measured in solution. A purified sample of UCNPs coated with azide-PEG-neridronate was measured and the relative absorbance was inserted into the linear equation obtained from the calibration.

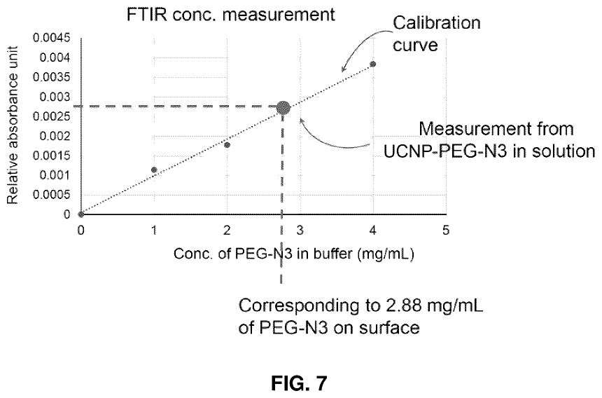

Results of this determination are shown in FIG. 7. An azide-PEG:UCNP ratio of 1.2 was calculated.

### Example 6

### Confirmation of Conjugation

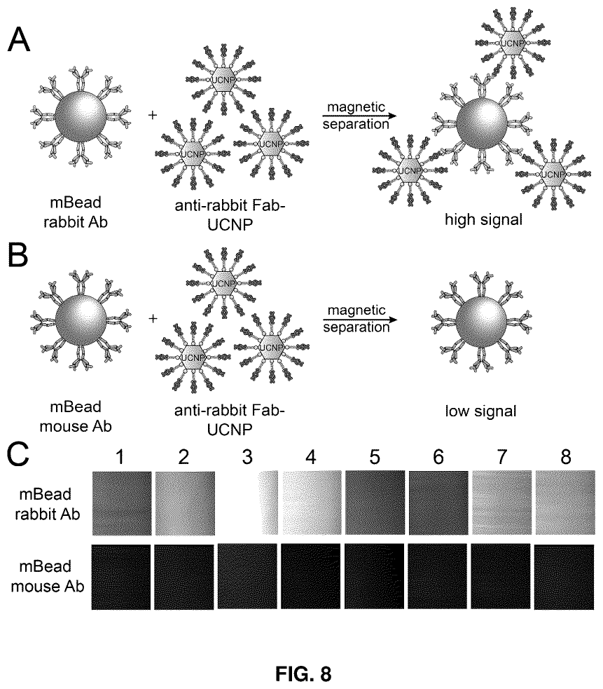

A magnetic bead assay was developed to confirm the successful conjugation of UCNPs with Fab antibodies, as illustrated in FIG. 8. As shown in this figure, A) magnetic beads were coated with a rabbit IgG antibody and incubated with anti-rabbit Fab conjugated UCNPs and form an immunocomplex with anti-rabbit UCNPs-Fabs. By applying an external magnetic field, unbound UCNP-Fab conjugates are separated from the sample. B) Control experiment using magnetic beads coated with a mouse IgG antibody. Here, the anti-rabbit UCNP-Fab conjugates cannot bind and are removed by the magnetic separation. C) Magnetically purified samples (1-8) are loaded into a glass capillary and imaged with an upconversion microscope. Higher intensities in the upper row and low intensities in the control (lower row) confirm that the anti-rabbit UCNP-Fabs bind specifically to the rabbit antibody-coated magnetic beads.

### Example 7

### Determination of Fab-UCNP Diameter

The hydrodynamic diameter of two Fab-UCNP constructs that have different UCNP cores were determined using Dynamic Light Scattering (DLS).

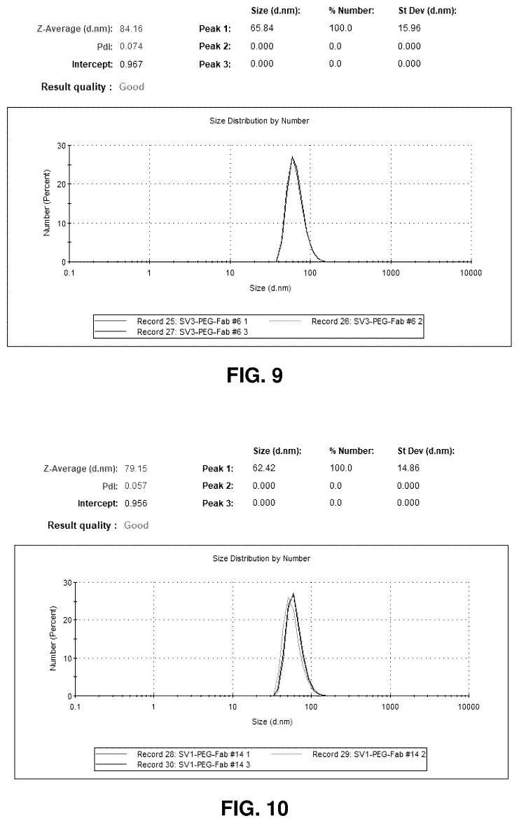

In FIG. 9 there are shown results for anti-mouse Fab conjugated UCNPs (Er-doped UCNP), showing that the constructs have a hydrodynamic diameter of 84.16 nm with a polydispersity index of 0.074.

In FIG. 10 results are shown for anti-mouse Fab conjugated UCNPs (Tm-doped). Here the constructs were shown to have a hydrodynamic diameter of 79.15 nm with a polydispersity index of 0.057.

1. A nanocomposite comprising:
an upconversion nanoparticle (UCNP), which comprises a surface coating having at least one conjugation anchor point; and
at least one functionalized antibody fragment, wherein the functionalized antibody fragment is covalently bound to the surface coating via the at least one conjugation anchor point.

2-30. (canceled)

31. The nanocomposite of claim 1, wherein the surface coating renders the upconversion nanoparticle water-dispersible.

32. The nanocomposite of claim 1, wherein the functionalized antibody fragment is functionalized via at least two adjacent thiol groups.

33. The nanocomposite of claim 1, wherein the functionalized antibody fragment is functionalized with a bifunctional reagent that on one end has a functional moiety that is connected to two adjacent thiol groups and at the other end has a second functional moiety that is covalently bound to the surface coating via the at least one conjugation anchor point.

34. The nanocomposite of claim 33, wherein the bifunctional reagent is a bis-sulfone-containing reagent.

35. The nanocomposite of claim 1, wherein the surface coating comprises at least one linker, which is at least bifunctional and that is coordinated to the UCNP.

36. The nanocomposite of claim 35, wherein the at least one linker is at least partly hydrophilic.

37. The nanocomposite of claim 35, wherein the at least one linker comprises at least one coordinating moiety.

38. The nanocomposite of claim 35, wherein the at least one linker comprises a phosphonate or bisphosphonate decorated (PEG)ₙ, where n=3-150.

39. The nanocomposite of claim 1, wherein the covalent binding of the functionalized antibody to the conjugation anchor point comprises azide-alkyne cycloaddition conjugation between a free azide group and an alkyne-containing group selected from BCN, COMBO, DBCO/DIBAC, BARAC, DACN, DIFO, DIFO2, DIFO3, DIMAC, ALO, NOFO, CliCr, MOFO, or OCT.

40. The nanocomposite of claim 1, having the general formula: 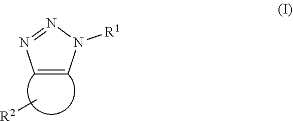
wherein:
R² represents antibody fragment-(PEG)ₙ, wherein n=1-12, and R³ represents UCNP-Y-(PEG)ₙ, wherein Y is a phosphonate or bisphosphonate group and n=3-150, wherein the ring structure to which R² is attached is an optionally substituted cyclic or heterocyclic ring structure containing from 5 to 9 atoms; or wherein R² represents UCNP-Y-(PEG)ₙ, wherein n=1-12, and Y is a phosphonate or bisphosphonate group, and R³ represents antibody fragment-(PEG)n, wherein n=3-150, wherein the ring structure to which R² is attached is a substituted cyclic or heterocyclic ring structure containing from 5 to 9 atoms.

41. The nanocomposite of claim 1, having the general formula: 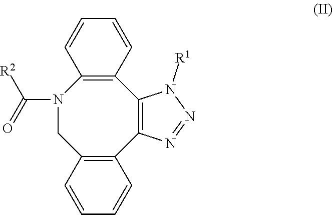
wherein:
R² represents antibody fragment-(PEG)ₙ, wherein n=3-12, and
R¹ represents UCNP-Y-(PEG)ₙ, wherein Y is a phosphonate or bisphosphonate group and n=3-150; or
wherein
R² represents UCNP-Y-(PEG)ₙ, wherein Y is a phosphonate or bisphosphonate group and n=3-150, and
R¹ represents antibody fragment-(PEG)ₙ, wherein n=1-12.

42. The nanocomposite of claim 1, wherein the antibody fragment contains one specifically reducible disulfide bond.

43. The nanocomposite of claim 1, wherein the antibody fragment is a Fab, Fab′, Fc, or pFc fragment.

44. The nanocomposite of claim 1, wherein the nanocomposite contains at least two different antibody fragments covalently bound to the surface coating.

45. A method for the preparation of a nanocomposite, the method comprising:
a. providing an upconversion nanoparticle comprising a multifunctional linker on its surface, the multifunctional linker having a free click reactive moiety;
b. reacting a reduced antibody fragment having at least one pair of adjacent interchain thiol groups with a thiol-reactive moiety, whereby the adjacent thiol groups react with the thiol-reactive moiety to form a functionalized antibody fragment; and
c. reacting the upconversion nanoparticle with the functionalized antibody fragment, thereby forming a nanocomposite having the functionalized antibody fragment covalently bound to the multifunctional linker.

46. The method of claim 45, wherein the thiol-reactive moiety is a bis-sulfone containing moiety.

47. The method of claim 45, wherein the linker is bifunctional.

48. The method of claim 45, wherein the linker is at least partly hydrophilic.

49. The method of claim 45, wherein the multifunctional linker is a phosphonate or bisphosponate decorated polyethylene glycol moiety having an azide group at its free end.

50. The method of claim 45, wherein the antibody fragment in step (b) is reduced using a reducing agent to reduce at least one exposed interchain disulfide bridge on the antibody fragment, thereby releasing adjacent interchain thiol groups for reaction with the thiol-reactive moiety.

51. The method of claim 45, wherein the thiol-reactive moiety in step (b) is a bis-sulfone-(PEG)ₙ-DBCO moiety, wherein n=3-12.

52. A method for the detection of a target material in a biological sample, comprising:
a. providing a biological sample;
b. contacting a nanocomposite comprising at least one upconversion nanoparticle linked to at least one functionalized antibody fragment with the biological sample, whereby the nanocomposite selectively binds to target material in the biological sample; and
c. obtaining an imaging signal from the nanocomposite thereby identifying the target material.

53. The method of claim 52, wherein the biological sample is a tissue sample or a blood sample.

54. The method of claim 53, wherein the biological sample is a tissue sample that has been fixed or wherein the tissue sample is frozen.

55. The method of claim 52, wherein the biological sample is contacted with a first nanocomposite for the detection of a first target material to obtain a first imaging signal, and wherein the biological sample, or a section from the same biological sample, is subsequently contacted with a second nanocomposite for the detection of a second target material to obtain a second imaging signal, wherein the first and second imaging signals are obtained by excitation at the same wavelength and detection at different wavelengths, and wherein optionally the first and second imaging signals are overlayed.

56. The method of claim 52, wherein the contacting with at least one upconversion nanoparticle in step (b) is preceded by a step of contacting the biological sample with at least one primary antibody, whereby the nanocomposite recognizes and binds to the primary antibody.

57. The method of claim 52, wherein the biological sample is counterstained using a conventional dye or stain prior to or after treatment with the nanocomposite and/or primary antibody.

58. The method of claim 52, comprising obtaining a first image by detecting the counterstain and obtaining a second image by detecting the upconversion nanoparticle, and optionally combining the two images.

59. The method of claim 52, wherein the nanocomposite is as set forth in claim 1.
